(19) United States
(12) Patent Application Publication
(10) Pub. No.: US 2007/0141683 A1
(43) Pub. Date: Jun. 21, 2007

(54) Selective electrode for benzene and benzenoid compounds

(76) Inventors: Lisa R. Warner, Boise, ID (US); Dale D. Russell, Boise, ID (US)

Correspondence Address: PEDERSEN & COMPANY, PLLC, P.O. BOX 2666, BOISE, ID, 83701, US

(21) Appl. No.: 11/600,674

(22) Filed: Nov. 15, 2006

Related U.S. Application Data
(60) Provisional application No. 60/737,070, filed on Nov. 15, 2005.

(51) Int. Cl. G01N 33/00; C12P 13/00

(52) U.S. Cl. 435/128; 436/104

(57) ABSTRACT

An apparatus may be adaptable for laboratory, field, or in vivo detection and measurement of benzene, benzenoids, or other organic molecules or compounds. A selective binding agent is created by binding a target molecule or similar molecule with an appropriate monomer, polymerizing the monomer, and removing the target or similar molecule. This procedure results in binding agent sites that are highly selective for the target molecule over other similar organic molecules. The finished binding agent is coated onto or otherwise incorporated with an electrode. The size of the electrode diameter may range from a scale of inches to a scale of sub micrometers, depending on the application. In preferred embodiments, polymers templated with a derivatized benzene molecule have shown to be effective even for detection and measurement of benzene, which is non-electroreactive. Benzene in a solution to which the templated polymer is exposed surprisingly results in increased conductivity of the polymer, with the conductivity increasing with increased benzene concentration in the solution.

This application claims priority of U.S. Provisional Application No. 60/737,070, filed Nov. 15, 2005, and entitled “Selective Electrode for Benzene and Benzenoid Compounds”, which is hereby incorporated by reference.

### BACKGROUND OF THE INVENTION

1. Field of the Invention

This invention relates generally to an electrochemical sensor for detecting organic compounds. More particularly, this invention relates to detection of benzene and benzenoid compounds using a highly selective sensor.

2. Related Art

There is significant need for a benzene-selective detector or benzenoid-selective detector. Benzene, a common industrial solvent, is a volatile organic compound (VOC) and carcinogen often found in discharge from factories, or in soil and water due to leaching from underground fuel storage tanks or landfills. Many other toxic or carcinogenic compounds contain a benzene ring, for example, catechol, which, when found in the environment, is often a sign that living organisms have been acting on benzene spilled or leached into the environment. Also, dopamine is a benzenoid compound of biochemical interest, but current methods of in vivo detection cannot distinguish it from ascorbic acid (Vitamin C).

Numerous sensors relying on a variety of molecular characteristics are employed to detect and/or measure the concentration of a substance in a given sample.

Russell (U.S. Pat. No. 5,244,562) discloses a switching device including an electrode coated with a templated polymer, wherein the switch is activated or inactivated depending on the concentration of glucose. This templated polymer electrode decreases current as glucose concentration increases.

Port, et al (U.S. Pat. No. 6,372,872 B1) discloses the formation of a rigid polymer that is selective for chosen dissolved ions. The monomer is complexed with the chosen ion prior to polymerization. After polymerization, the ion is then removed and the remaining polymer processed and coated on a substrate. The polymer may be coated onto an electrode or similar device for use in a detector. The Port, et al. method does not disclose or teach a method for detecting organic molecules such as benzene. Further, Port, et al. reports difficulties in coating the templated polymer without significant loss of active binding sites.

Russell (U.S. Pat. No. 6,436,259 B1) discloses an electrode that is selective for mercury. It uses a chelating agent that is covalently bound to a polymer to bind mercury ions from a solution. The binding agent is coated onto an electrode to build a detecting and measuring device for ions, but not for organic compounds.

The inventors believe there are no methods and apparatus in the prior art for electrochemical detection and measurement of benzene. Benzene is known not to be electroreactive, that is, benzene does not undergo electron transfer reactions in aqueous solution at solution-accessible electrical potentials. Also, the simple cyclic structure of benzene, without any groups (only hydrogen) bound to its carbons, has no chemical “anchors” or “hooks” that are needed for binding for electrochemical analysis. Therefore, benzene is not expected to be detectable or measurable by electrochemical means. The present invention, however, surprisingly provides an electrochemical apparatus and method for measuring benzene.

### SUMMARY OF THE INVENTION

The invented device comprises an electrochemical sensor used to detect organic molecules and compounds, and, more specifically, benzene and benzenoid compounds in liquid and gas phases. The device includes a sensor comprising an electrode that is coated with, or that otherwise comprises, a templated polymer that selectively binds with a benzene molecule(s) or benzenoid compounds. When the sensor is placed in contact with a solution or gas phase containing the target benzene or benzenoid molecule/compounds, the target benzene/benzenoid will bind with the active templated sites on the polymer, changing the conductive properties of the resulting polymer complex in a manner that may be correlated to the concentration of the benzene/benzenoid.

Benzene is not electroreactive, and, hence, benzene analyte from a liquid or gas being tested would not be expected to exhibit electron transfer when captured/bound in the templated site and subjected to a potential. Thus, conductivity of a polymer or a “molecular imprint polymer (MIP)” would not be expected to increase with benzene concentration in a solution being analyzed. Still, the presence of benzene analyte at the templated sites in the polymer surprisingly has been found to increase conductivity of the polymer. The inventors believe that the active templated sites on the polymer may be considered “holes” in the polymer, and, when benzene analyte molecules fill the templated sites, the benzene molecules act as “switch-closing” molecules in the “circuit” of the polymer. This, the inventors believe, allows flow of current across the previously-vacant, benzene-filled holes in the polymer, even though benzene is not electroreactive. For electroreactive analytes, for example, catechol, it is believed that the analyte itself contributes electron transfer, and, hence, will increase current flow by virtue of its presence by contributing electron transfer, in addition to “closing the switch” of the templated site.

The preferred templated polymers comprise active binding sites that are created by esterification, before polymerization of a selected monomer, of a benzenoid compound derivatized with carboxylic acid groups or acid chloride groups. The ester is formed by acid-base chemistry between the derivatized benzenoid compound and preferably a plurality of monomers having basic sites, such as amine sites. The benzenoid compound is then removed from the polymer by reversing the esterification, for example, by a mild acid or mild base wash.

An object of the preferred embodiments of the invention is to provide electrochemical devices and methods for detecting and measuring benzene. Another object is to provide a robust, firm, templated polymer that may be incorporated into a probe or sensor for detection of an organic analyte, preferably benzene and benzenoid compounds, in an aqueous or gaseous environment. Another object of some embodiments is to provide benzene and benzenoid detection and measurement apparatus and methods for environmental study and cleanup and/or for biochemical study and diagnosis, preferably even at parts per billions levels and even for in vivo study and diagnosis. Another object of the preferred embodiments is to provide such a probe or sensor that may be made to be very small for cellular level testing, for example, on the order of 10⁻⁶ meters.

### BRIEF DESCRIPTION OF THE DRAWINGS

FIG. 1A is a schematic diagram of one embodiment of a sensor according to the present invention, incorporating one embodiment of the templated polymer for analyzing an analyte in aqueous solution.

FIGS. 1B and 1C are schematic diagrams of another embodiment of a sensor according to the present invention, using a thin, paddle electrode, wherein FIG. 1C is a cross-section showing the pre-coat layer and the templated polymer layer.

FIG. 2 illustrates steps in a synthesis of one embodiment of a benzene-monomer complex according to the invention.

FIG. 3 illustrates further steps in the preferred synthesis, comprising polymerization of the benzene-monomer complex with thiophene dimer.

FIG. 4 illustrates further steps in the preferred synthesis, wherein the templating molecules of the polymer of FIG. 3 are removed from the benzene-polymer-complex to produce one embodiment of a benzene-selective templated polymer according to the invention.

FIG. 5 schematically illustrates a synthesis according to an alternative embodiment of the invention, wherein derivatized cyclopentane dithiophene is reacted with the benzene templating molecule to arrive at an alternative monomer-template complex.

FIG. 6 schematically illustrates polymerization of the monomer-template complex of FIG. 5 with cyclopentane dithiophene, and the subsequent removal of the templating molecules.

FIGS. 7A and 7B schematically illustrate alternative syntheses using boroester and amine chemistry, respectively.

FIG. 8 is a worked example data graph, which shows the significant response of an electrode, coated with a templated-polythiophene according to one embodiment of the invention, to solutions containing zero benzene and 10 ppm benzene.

FIG. 9 is a worked example data graph, which shows little or no response of a bare-metal electrode to solutions containing zero benzene and 10 ppm benzene.

FIG. 10 is a worked example data graph, which shows little or no response of an electrode coated with poly-bi-thiophene, without templating, to solutions containing zero benzene and 10 ppm benzene.

FIG. 11 is a worked example data graph, which shows the response of an embodiment of a benzene-selective electrode/sensor comprising a templated polymer made according to the synthesis of FIGS. 2-4, to a blank (0.1 LiClO₄ in DI H₂O), and to a titration of benzene in 0.1 LiClO₄ in DI H₂O, graphed as I (amps/cm²) vs. E (volts), over a range of about −1 volt to +1 volt.

FIG. 12 is an exploded view of the data, from −0.4 to 0 volts, from FIG. 11.

FIG. 13 is a graph of the current at −0.45 volts vs. concentration of benzene (ppb), using data from the testing represented by FIGS. 11-12.

FIG. 14 is a graph of current at −0.45 volts vs. Log [Benzene concentration, ppb], using data from the testing represented by FIGS. 11-13.

FIG. 15 is a graph of the response, of a benzenoid-selective electrode-sensor comprising a templated polymer made according to the synthesis of FIGS. 2-4, to various concentrations of toluene in 0.1 LiClO₄ in DI H₂O, wherein current at −0.45 volts vs. toluene concentration (ppb) is plotted.

FIG. 16 shows the current (A) vs. potential (volts) response, of another benzenoid-selective electrode-sensor comprising a templated polymer made according to the synthesis of FIGS. 2-4, in a blank of 0.1 LiClO₄ in DI H₂O, and also in two solutions, one with a low concentration of catechol in 0.1 LiClO₄ in DI H₂O, and one with a high concentration of catechol in 0.1 LiClO₄ in DI H₂O.

### DETAILED DESCRIPTION OF THE PREFERRED EMBODIMENTS

Referring to the figures, there are several, but not the only, embodiments of the invented electrochemical sensor and syntheses for embodiments of the invented benzene and/or benzenoid-selective templated polymer. Also, there are shown several, but not the only, methods for using an embodiment of benzezoid-selective templated polymer according to the invention.

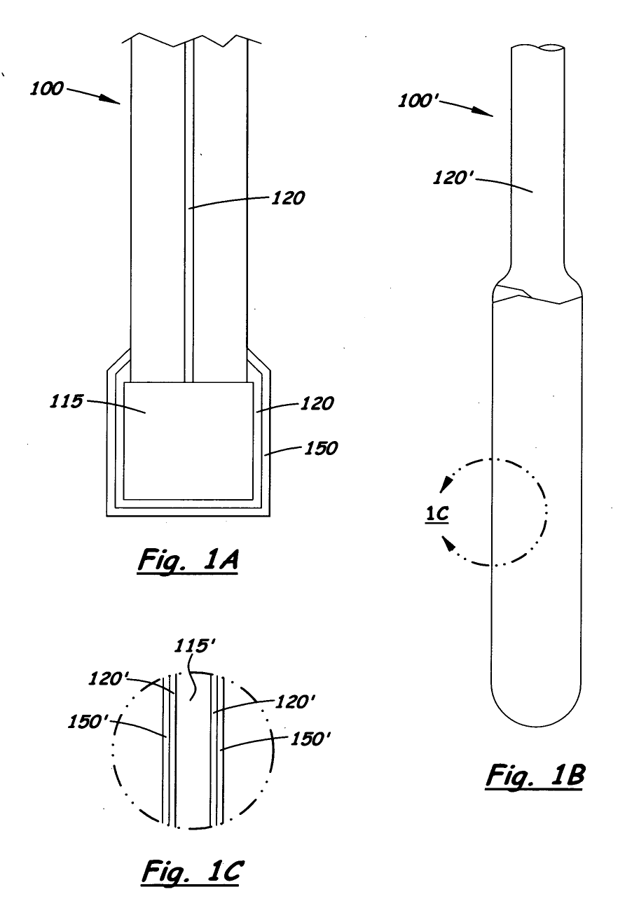

As shown schematically in FIGS. 1A 1C, the preferred electrochemical sensors use an electrode coated with at least one layer of polymer to detect and measure the presence of a target organic analyte in a solution. Coating the exterior surface of a probe is an effective method according to embodiments of the invention, but other methods of incorporating a templated polymer into a probe or other sensor may be used.

FIG. 1A illustrates a sensor 100 comprising a conductive electrode 115 for electrically communicating with known amperometric apparatus via connection 120. The electrode 115 is preferably pre-coated with pre-coat layer 120, such as a bi-thiophene polymer layer, that does not comprise templating. A bi-thiophene polymer pre-coat layer is preferred because bi-thiophene polymerized/plates at a lower potential than polythiphene, thus tending to prevent oxidizing/sintering. A single coating layer of pre-coat and a single layer of templated polymer are preferred, but, alternatively, multiple layers of pre-coat and/or multiple layers of templated polymer may be used.

The pre-coated electrode is then coated with a templated polymer 150 such as a preferred benzene-selective polymer as further described below. FIGS. 1B and 1C illustrate an alternative sensor 100′, which is generally a thin, flat paddle-shape 115′ and which is also pre-coated with a bi-thiophene layer 120′ and then coated with a templated polymer 150′ according to embodiments of the invention. The paddle electrode has been found particular effective, as embodiments may easily be made without seams or incongruities that may effect measurements. With such apparatus, one may expose the templated polymer, as an outer surface of an electrode, to the aqueous solution, and analyze for benzene and/or benzenoid compounds by potentiometric, voltametric, amperometric, or conductimetic means.

The templated polymer is made selective for the target analyte by reacting the desired monomers with the templating molecule, which may be the target analyte molecule/compound or an “analyte-surrogate” or “analyte-analog,” that is not the target analyte but rather a molecule/compound that has size, shape, composition, and/or electrostatic properties similar to the target analyte. Hereafter, the term “molecule” is used for simplicity, but it is to be understood that compounds, and a mixture of various molecules/compounds, may also be effective as templating units and as analytes in the devices and methods of the invention.

Preferably, at least the portion of the analyte-surrogate chemical structure that bonds to the monomer is similar to the target analyte in composition and electrostatic properties. Optionally, a mixture of the target analyte and surrogate analytes may be reacted with the desired monomers. Optionally, more than one monomer may be bound to multiple sites of a single templating molecule, such as is discussed below for ethylenediaminetetraacetic acid (EDTA).

Preferably, once templating molecules and monomers are bound together to result in what may be hereafter referred to as the “monomer-template complex,” the next step is polymerization of the monomer-template complex, or, optionally polymerization of the monomer-template complex together with monomers not bonded to templating molecules. In preferred embodiments, the polymer resulting from this polymerization features templating molecules covalently bound at multiple sites in the polymer chain that are not necessarily next to each other. This spacing of templating molecules may be managed by controlling the ratio, present in polymerization, of monomer-template complex compared to monomer not bonded to templating molecule(s). Or, the spacing of templating molecules may be managed by providing a templating molecule that comprises multiple bonding sites for monomer, and that itself distances the multiple monomers bound to it by virtue of its own chain lengths or other “spacer arms.” In this second scenario, when the templating molecule is removed after polymerization, as discussed further later in this disclosure, the multiple sites that were previously bonded to the templating molecule will now be relatively far distanced from each other in regions of the polymer that are no longer connected by the templating molecule but that have polymerized, and cross-linked, to other portions of the large network of polymer structure.

An illustration of how a spaced arrangement of active templated sites may be achieved may be shown by an example involving ethylenediaminetetraacetic acid (EDTA), which has four basic reactive sites and which may bind to four monomers. For example, monomer molecules may be derivatized to have acidic functional groups, and then four monomers with their acidic functional groups may be reacted with the four basic reactive sites of EDTA to form a “super-molecule.” When added to a polymerization system comprising more monomer (with or without more EDTA), the original EDTA-bonded monomers become part of long, wrapped, and/or cross-linked chains that form a complex network. When the EDTA is removed, the original monomers' derivatized functional groups, previously bound to the EDTA, are exposed at relatively far distant locations in the network of polymer, no longer connected by the EDTA.

In many embodiments, an excess of monomer is present during the step(s) of bonding templating molecule to the monomer, and an excess of monomer is typically maintained in the solution prior to polymerization. This will typically result in templating molecules being located at multiple, fixed, but random or semi-random positions in the chain. Thus, the resulting polymer fixes the template sites at multiple locations in the polymeric structure, and the templating molecules may then be removed without altering the polymer geometry, except, as discussed above, that the connection between multiple chain portions (that is afforded by a templating molecule bonded to multiple monomers) may be broken upon removal of the templating molecule.

Polymerization may be done utilizing, or in the presence of, an electrode or an electrode pre-coated with polymer not having any templated sites, for example. This way, the polymer comprising templating molecules is attached to the electrode or the pre-coated electrode at the time of polymerization. Galvanometric solution polymerization at a potential wherein the monomer is electroreactive (without oxidizing the polymer) is preferred, but other polymerization methods may be used, for example, any potentiostatic control of potential during polymerization and deposition of the polymer, spin-coating, vapour deposition, Langmuir-Blodgett, or others.

Alternatively, but less preferably, the polymerization of the polymer comprising templating molecules may occur separated from the electrode, and the polymer may be later attached to the electrode in a separate step either before or after removal of the templating molecules.

Once polymerization has taken place, the next step preferably is removing the templating molecules from the polymer, to leave “holes” in the polymer that act as active sites which molecules of similar size, shape, and electrostatics may occupy. Each “hole” preferably maintains its shape, size, and electrostatic characteristics for an extended time, for example, for at least 50 uses, and preferably at least 1 year, because the polymer around each hole retains its firmness and rigidity for at least that amount of time. The characteristics of each “hole,” therefore, result from the characteristics of the surrounding polymer and the templating molecule/compound (that has since been removed), or at least, the portion of the templating molecule that reacted with the monomer. In cases wherein a mixture of different templating molecules is used, such as target analyte mixed with analyte surrogates, the holes left by removal of the multiple, different templating molecules still are expected to very similar or identical, as bonding of the different templating molecules to the monomers is expected to involve similar covalent bonding sites. In cases wherein multiple monomers react with multiple sites of a single templating molecule, the removal of that molecule may leave multiple sites exposed that are the same to the extent that the sites on the templating molecule were the same and to the extent that the polymer surrounding each site is the same. As discussed above for the EDTA example, the monomer sites that were once bonded to a single templating molecule may be far distant in the polymer and hence may have in its surroundings different cross-linking or other features. The preferred embodiments of the invention comprise a templated polymer that selectively binds, complexes, or otherwise captures benzene, even though it is not electroreactive and does not have the chemical “hooks” or “anchors” that are expected to be necessary for electrochemical analysis.

Preparation of benzene-selective templating sites is done by templating the polymer with a molecule(s) comprising benzene derivatized with active groups that may be bound to a selected monomer preferably before polymerization. After polymerization and after subsequent removal of the templating molecule/compound from the polymer, active templated sites remain that are selective to target analytes that are the same or that resemble the original templating molecule/compound. Even through benzene (C6H6) is not the templating molecule in the preferred embodiments, the inventors have found that the preferred polymers templated with derivatized benzene may be made that are selective to benzene (C6H6). The inventors believe that, depending upon the components and conditions selected for polymerization, and optionally, upon post-polymerization treatments, the templated polymer may be more or less selective to benzene vs. larger benzenoid compounds, as discussed later in this Detailed Description.

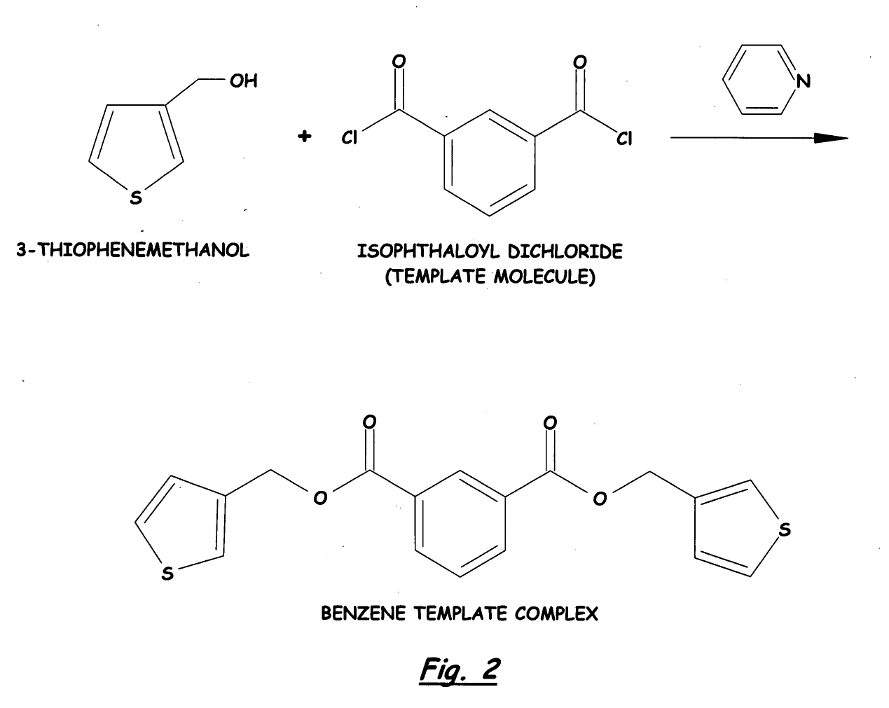

A preferred synthesis of a benzene-selective polymer includes use of a benzene compound comprising a plurality of derivatized sites, for example, at the 1 and 3 carbons of the benzene ring. FIG. 2 illustrates one embodiment of such a templating molecule: isophalaloyl dichloride, which comprises a benzene ring with Cl—C═O groups substituted onto two of the benzene carbons in meta relationship. By utilizing a benzene with two substituted groups, the benzene molecule may be bound to a plurality of monomer units, and may ultimately be incorporated into the polymer bound to portions of polymer on at least “two sides” of the templating molecule. Then, removal of the templating molecule will truly leave a “hole” in the polymer that has substantial structure around it, said substantial structure being likely to be more rigid and to have chemical and electronic characteristics that may be very selective in receiving subsequent analytes.

Other the other hand, if the templating molecule were attached at one site/carbon on, or near the benzene ring, this would result in the templating molecule being bound to one monomer unit, and incorporated into the polymer with polymer only on “one side” of the templating molecule. The templating molecule would then, in effect, extend from the surface of the polymer, generally as a branch off of the bulk of the polymer rather than being imbedded in it. Removal of such a templating molecule would not leave a “hole” in the polymer, or, at least, would not leave a hole with substantial structure around it, and, hence, would not be very selective to the target analyte.

The synthesis shown in FIG. 2 binds the templating molecules with monomer via esterification to form a monomer-template complex, which in this case may be called a benzene-monomer complex. As illustrated in FIG. 2, 3-thiophenemethanol and isophthaloyl dichloride (an acid chloride) are reacted in the presence of pyridine (an organic base believed to scavenge for HCl) to form an ester, R—OOC—C6H4—COO—R, wherein R=3-methylthiophene. Note that esterification takes place on “two sides” of the benzene ring, that is, at two derivatized groups (COCl or Cl—C═O) that are in meta positions on the benzene ring, resulting in the benzene ring being generally centered in the complex. Meta or para attachments of the benzene ring to the thiophene are preferred, as this “imbeds” the benzene ring in the benzene-monomer complex.

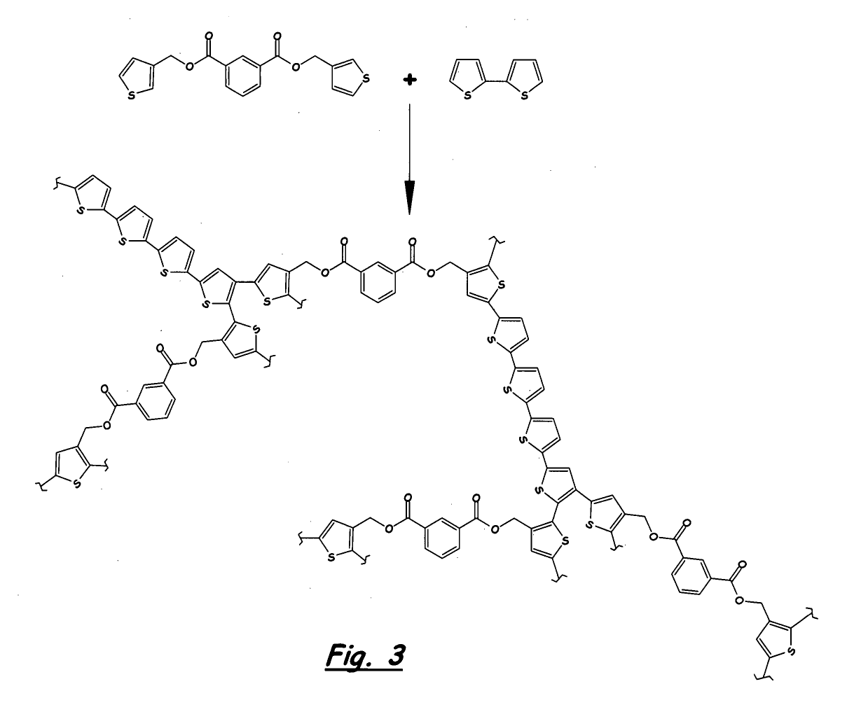

FIG. 3 illustrates one mode of polymerizing a monomer-template complex, resulting in a polymer-plus-templating molecule that comprises many benzene rings imbedded in the polymer, each being bound on “two sides.” The polymerization is conducted by adding the benzene-monomer complex (here, an ester) resulting from FIG. 2 to bi-thiophene dimer, all in a polymerization solution comprising acetonitrile, nitromethane, or another solvent, for example. Polymerization may be conducted at about 0-10 degrees C. and with ratios of bi-thiophene dimer to benzene-monomer complex (the ester) preferably in the range of about 3:1 up to about 5:1. The preferred method polymerization comprises conducting the polymerization in the presence of a platinum electrode that has previously been coated with a layer of polythiophene (polymerized bi-thiophene) that does not comprise templating molecules.

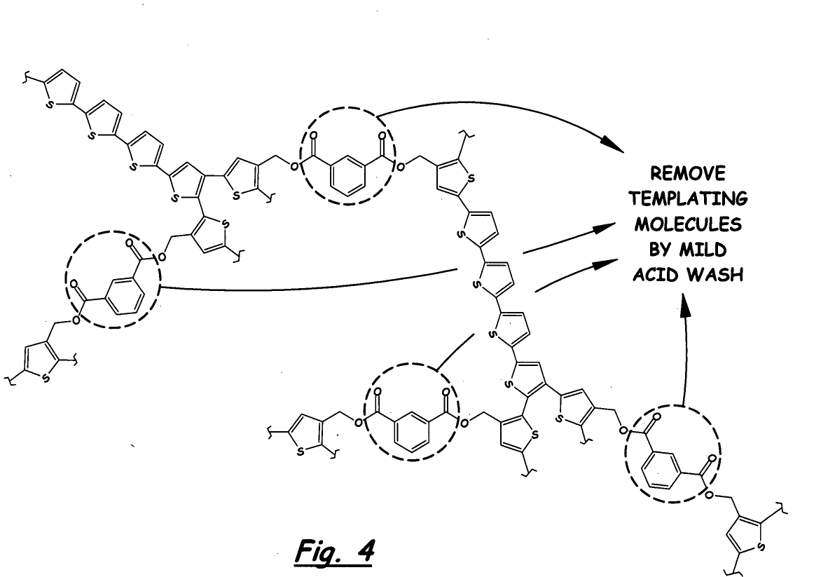

FIG. 4 illustrates one mode of removing the templating molecule from the polymer, leaving “holes” in the polymer for binding, complexing, or otherwise capturing benzene and/or benzenoid compounds, in other words, benzene or “benzene surrogates.” The preferred method of separating the templating molecule from the polymer comprises altering the pH of the solution, to reverse esterification by hydrolysis. As illustrated in FIG. 4, a mild acid wash using dilute HCl breaks C—O single bonds to free the acyl groups of the derivatized benzene, thus cleaving the templating molecule out of the polymer by the acid-base chemistry. Alternatively, acetic acid or other non-oxidizing mild acid may be used, preferably at room temperature. Alternatively, a mild basic wash may be used to cleave the templating molecules out of the polymer. In either the acid or base wash, a high volume of material is flushed across the polymer, to drive the equilibrium in the direction that remove the templating molecule from the polymer.

After cleaving the templating molecules out of the polymer, the polymer remains at, or very close to, its pre-templating-molecule-removal rigidity level and form and structure, except for the holes left by said removal/cleavage. This rigidity holds the active templated binding sites, now exposed by removal of the templating molecule, in position for the target analyte to react with them and become bound. This results in the formation of a template, at each exposed site, that a molecule of similar size, shape, and electrostatics may occupy. In other words, a template is left that is highly selective for the target analyte, minimizing the occurrence of false positive results.

The active templating sites left by the synthesis shown in FIGS. 2-4 are especially-selective to benzene and benzenoid compounds that comprise a single ring, for example, toluene and catechol, as shown below in Worked Examples 1 and 2, below. This templated polymer and others synthesized using a single-ring templating molecule are expected to be selective to benzenoid compounds that have a single ring, or even multiple, fused rings, but probably only if a ring is “exposed” on an end of the compound for bonding/complexing with the templating site.
For example, it is expected that a molecule such as 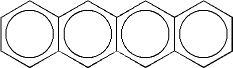
may fit into the templating site, but that a molecule such as 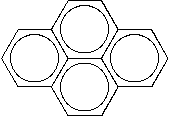
may not fit into the templating site.

If the templated polymer “relaxes” during or after the templating molecule cleavage, the active sites may become less selective because other molecules may fit into the template “holes.” Therefore, the effectiveness and selectively of templated polymers according to some embodiments of the invention may be adjusted/controlled by increasing the rigidity of the templated polymer, so that its rigid structure tends to hold firmly a substantially unchanging “hole” for capturing a particular species. Further, the selectivity also may be adjusted by tightening the templated sites or otherwise restricting access to the templated sites. For example, cross-linking of the polymer may help improve polymer rigidity to either maintain a desired site size and characteristics or to tighten/shrink the site to obtain alternative site size and characteristics. However, one does not desire so much cross-linking that the templating molecule cannot be removed from the polymer during the steps described above or so much that analyte molecules will be unable to access the sites. Also, radiation, or other excitation of the polymer, for example, by laser or other means, may increase rigidity of the polymer. Adding energy to the polymer by radiation or other means may move the polymer along its energy curve, over an “activation energy” peak, to a lower energy state as a more rigid, typically more twisted, configuration, wherein the “holes” left by the templating molecule are typically tighter and less prone to relax.

Modifying the selectivity of the preferred templated polymers may be desirable for increasing selectivity for benzene over other benzenoid molecules. When two or more of the benzenoid compounds are present, embodiments of the sensor made according to the methods in FIGS. 2-4 tend to detect all the benzenoid compounds present, yielding a “total benzenoid” signal, in effect, rather than a signal that differentiates them. For example, in the polymer of FIG. 4, due to the original size of the templated site (due to removal of HOOC—C6H4-COOH) and due to the polar nature of the templated sites of the polymer (comprising oxygen after cleavage of the templating molecule), the templated polymer may exhibit selectivity for catechol (with its polar OH groups) that is slightly greater than its selectivity for toluene (with its CH3 group), which in turn is slightly greater than its selectively for benzene (simply C6H6). In other words, these sensor embodiments have a differential capacity to bind the three compounds in the order of catechol, greater than toluene, greater than benzene. This is acceptable in many testing environments. For environments where high selectivity to benzene in the presence of toluene, catechol, or other similarly-sized benzenoids is desired, however, further methods according to alternate embodiments of the invention may include “tightening” the templated site to modify selectivity toward benzene. For example, steps that increase polymer rigidity and tighten the templated site, such as discussed above, may improve benzene selectivity; these steps may lessen the tendency of the templated sites to enlarge or “loosen,” during or after the templating molecule cleavage, to become more accessible to larger analyte molecules, and/or these steps may actually “shrink” the sites and make them less accessible to the larger toluene and catechol molecules.

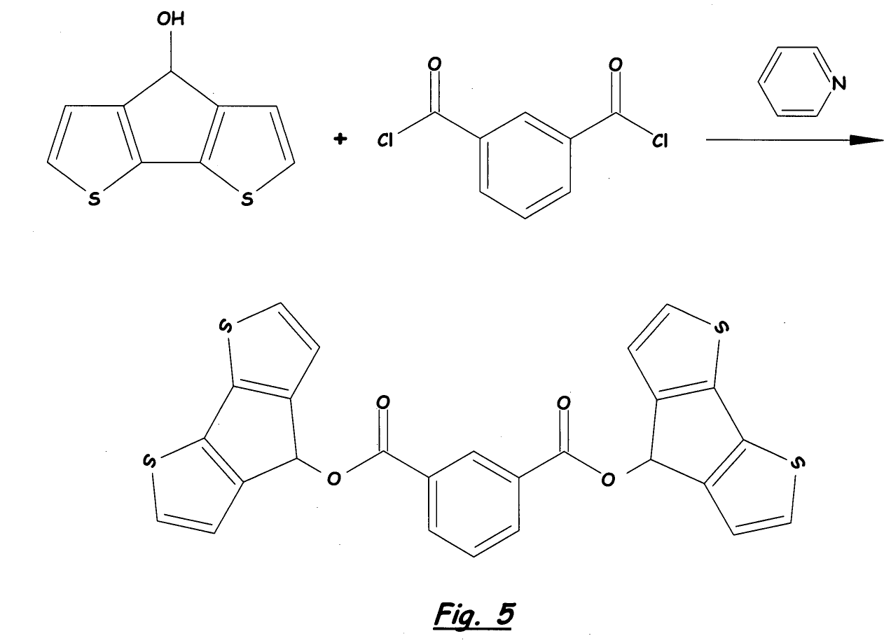

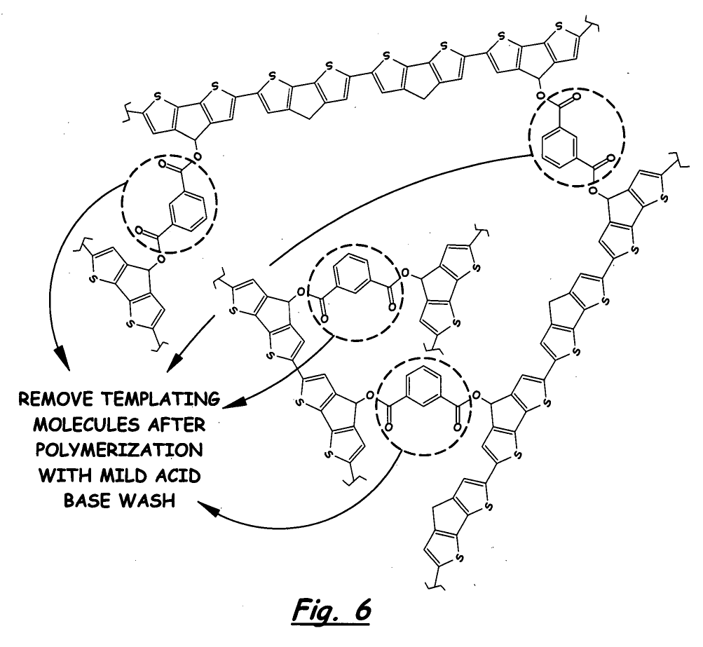

A synthesis of benzenoid-selective templated polymer according to alternative embodiments of the invented are envisioned to include the cyclopentane dithiophene (CPDT) monomer rather than, or in addition to, the 3-thiophenemethanol of FIG. 2. One synthesis of a CPDT monomer-template complex is shown in FIG. 5, wherein CPDT derivatized with OH is reacted with isophthaloyl dichloride to “center” the benzene between two CPDT molecules. The monomer-template complex is then polymerized with an excess of CPDT, after which the templating molecule is removed by a mild acid or base wash, resulting in a templated polymer schematically portrayed in FIG. 6. This templated polymer is expected to exhibit excellent selectivity to benzene, due to the benzene-selective templated sites and due to the increased polymer rigidity expected from the three-ring CPDT monomer. The three-ring monomers are expected to maintain structures on each side of each templating site that are less likely to relax than thiophene units, thus helping to prevent loosening of the templated sites between the CPDT molecules.

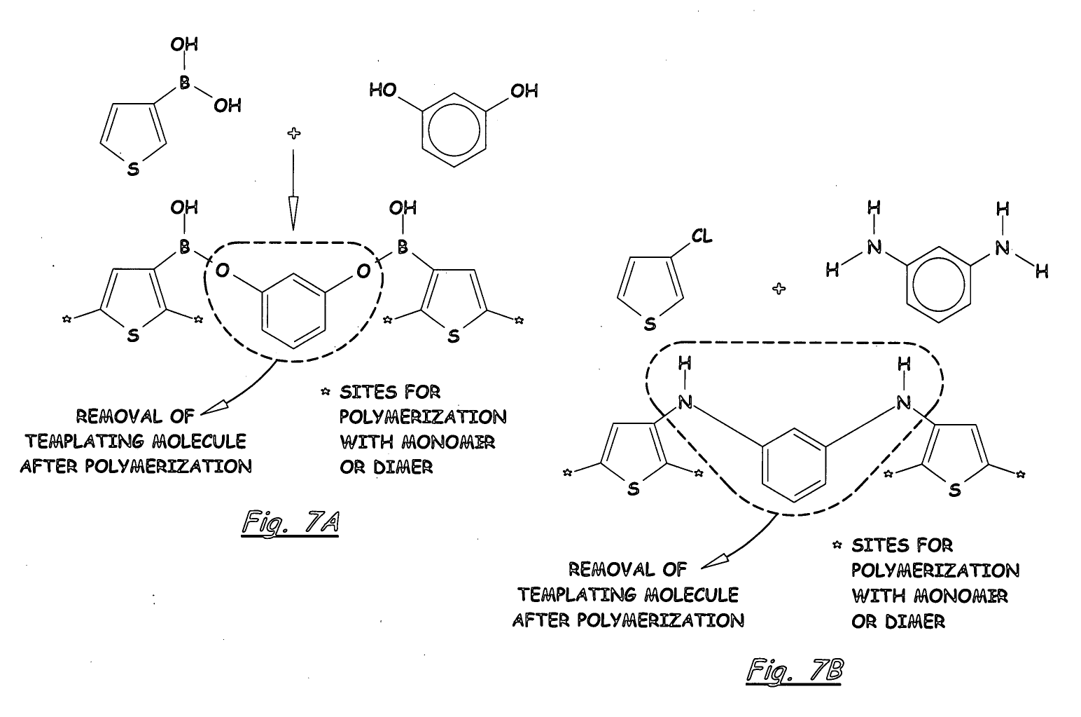

As discussed above, methanol-derivatized rings, such as 3-thiophenemethanol, may be used with an acid chloride in esterification as a path to the templated polymer. Alternatively, other syntheses may be used, including any reversible-equilibrium chemical reaction forming a covalent bond between the templating molecule (preferably comprising a single benzene ring) and a polymeric backbone (preferably, a thiophene or biothiophene-based polymer backbone). Acid-base chemistry, hydrogen bonding, condensation, or elimination paths may be used, for example. Boron chemistry may be used, for example, reacting a benzene ring derivatized with two hydroxyl groups with boron-derivatized thiophene, as shown schematically in FIG. 7A. Also, for example, amine chemistry may be used, for example, reacting a chloride-derivatized thiophene with di-amine-derivatized benzene via condensation, as schematically illustrated in FIG. 7B, which would be reversed by acid flushing.

The resulting sensor may be used for measuring even very low concentrations of benzene or benzenoid analyte, even in harsh environments. The conductive properties of the resulting electrode vary with the number of template sites that are become bonded to the target analyte. Thus, detection occurs by measuring any of several properties, including measurement of potentiometric, voltametric, amperometric, and conductimetic properties. For voltametric, amperometric, and conductimetic detection, any conductive or semi-conductive polymer is a candidate for templating. For potentiometric detection, the polymer may be conductive, semi-conductive, or an insulator, that is, the conductivity does not matter as the surface charges may be measured. In any event, however, the preferred polymer is derivatizable with a functional group that can enter into a reversible equilibrium with the target analyte or a target analyte surrogate. This permits the templating process to occur during synthesis of the bulk polymer, and the formation of one, and preferably many, re-useable binding site(s) for the analyte.

The preferred polymers are of thiophene type, such as polythiophene or poly-bi-thiophene or other derivatized polythiophenes. These polymers are preferred because they are semi-conductive and they do not swell or deform in the presence of water. Alternative polymers include polyacrylamides, polyacetylenes, polypyrroles, polyanilines, polythiofulvalenes, and many others, including numerous derivatized forms of each of these.

### WORKED EXAMPLES

Synthesis of the preferred monomer-template complex was completed and replicated to ensure repeatability. The success of each synthesis was verified with Fourier transfer infrared (FTIR) on a Mattson 6020 galaxy, proton and carbon NMR on a Varian Mercury 300 MHz NMR. Polymerizations using various concentrations of potential binding sites were completed using galvanometric solution polymerization on a Par EG&G 263A controlled with CorrWare software from National Instruments. To ensure that degradation of the monomer-template complex had not occurred during the polymerization process, reflectance FTIR spectra, taken on a ThermoNicolet Continuum, of each electrode were analyzed and key functional groups attaching the monomer to the template were identified.

### Example 1—Templated Polymer Sensor Measuring Benzene

1. A platinum electrode was pre-coated with poly-bi-thiophene by galvanometric solution polymerization.

2. A monomer-template complex was synthesized according to the method in FIG. 2.

3. Polymerization of this monomer-template complex with bi-thiophene dimer, as in FIG. 3 was then carried out, again by galvanometric solution polymerization.

4. Removal of the templating molecule from the polymer was done by raising pH with a wash of NaOH solution of approximately 10 pH for 2-3 minutes.

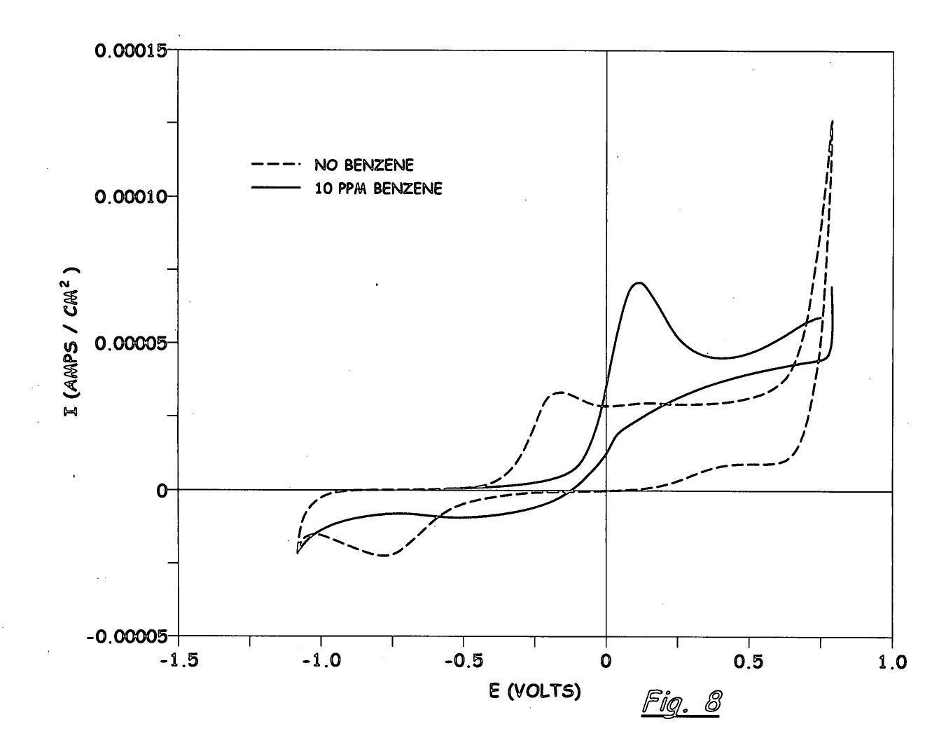

5. The resulting, templated-polymer-coated sensor was then tested by subjecting the electrode to a blank solution (no benzene) containing deionized water and electrolyte (NaClO4) during a cyclic voltammogram (reversible cyclic voltametric waves, amps/cm² vs. volts), and then repeating the test wherein the solution comprises 10 ppm benzene. The voltammogram results are shown in FIG. 8.

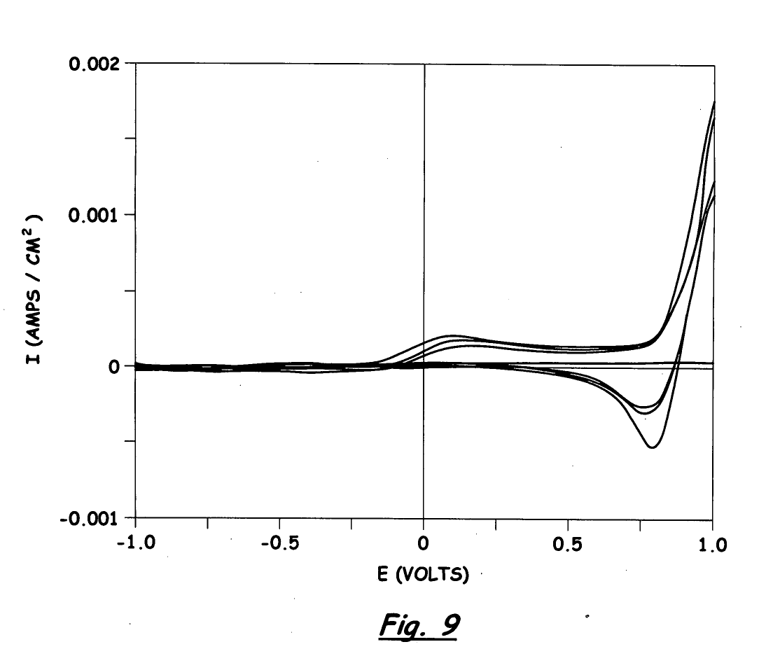

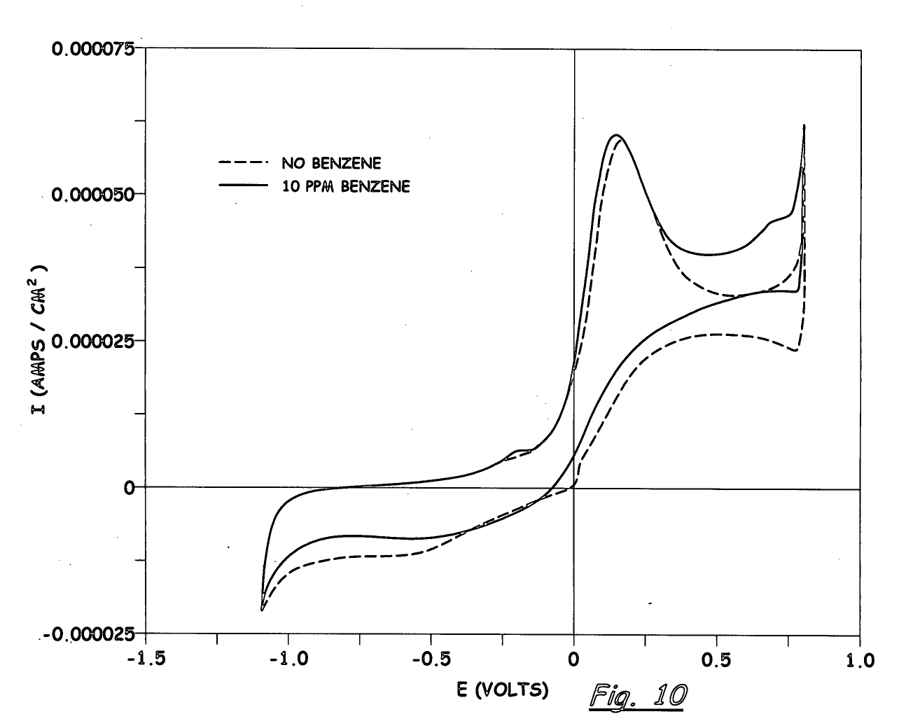

6. To confirm that results from the templated-polymer-coated sensor were not an effect of the platinum electrode or the poly-bi-thiophene, a base platinum electrode (FIG. 9), a poly-bi-thiophene-coated electrode (without any templating, FIG. 10) were tested in solutions made as in number 5 above. No significant response was seen in either the bare-electrode case or the poly-bi-thiophene case; while there are some differences in the data above 0.5 volts when these electrodes were exposed to no benzene and to 10 ppm benzene, these are believed to be due to other variables and not to benzene. Each of the bare electrode and the poly-bi-thiophene electrodes show almost identical responses to no benzene and 10 ppm benzene solutions in the range of interest from −0.5 volts to +0.5 volts, compared to the significant change in response from no benzene to 10 ppm benzene, in the range of −0.5 to +0.5 volts, by the templated-polymer electrode (FIG. 8).

### Example 2—Benzenoid-Selective Sensor Measuring Benzene, Toluene, and Catechol

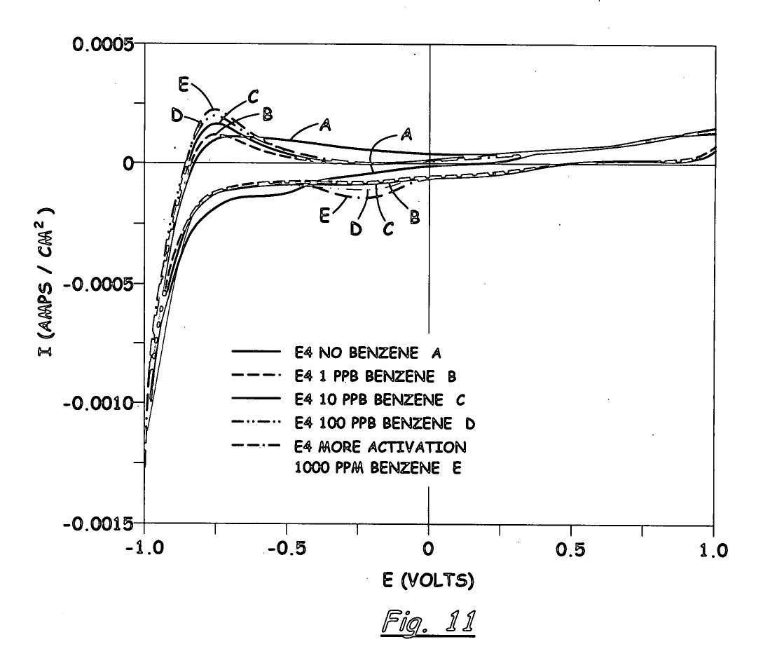

A sensor was constructed using the methods and materials as shown in FIGS. 2-4 and described earlier in this Detailed Description. This sensor (templated-polymer-coated electrode) was tested to determine how and if it responded to changing concentrations of benzene, toluene, and catechol. The results are shown in FIGS. 11-16.

FIG. 11 represents the response of the sensor to a blank and to a titration of benzene into 0.1 LiO₄ in deionized H₂O, by means of a CV curve run from 0 to −1 to 1 to 0 volts. Data is shown for the following concentrations: no benzene; 1 ppb benzene; 10 ppb benzene; 100 ppb benzene; and 1000 ppb benzene. One may note from FIG. 11 that there is a current increase in the no benzene data around −0.4 volts, whereas the 1 ppb-1000 ppb data shows stable and even decreasing current in that region.

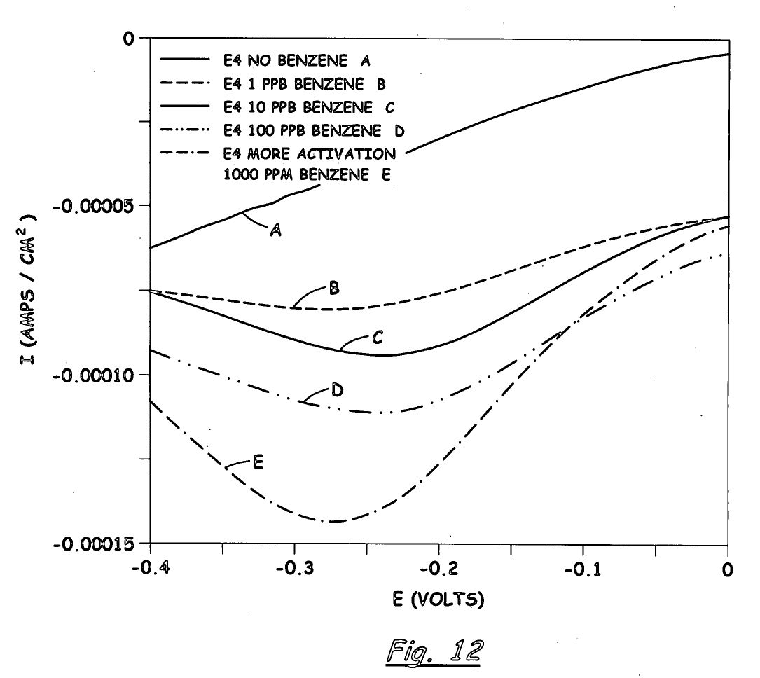

FIG. 12 shows the data of FIG. 11 plotted between −0.4 and 0 volts, so that one may see more clearly the differences between the data with no benzene and the data with various benzene concentrations.

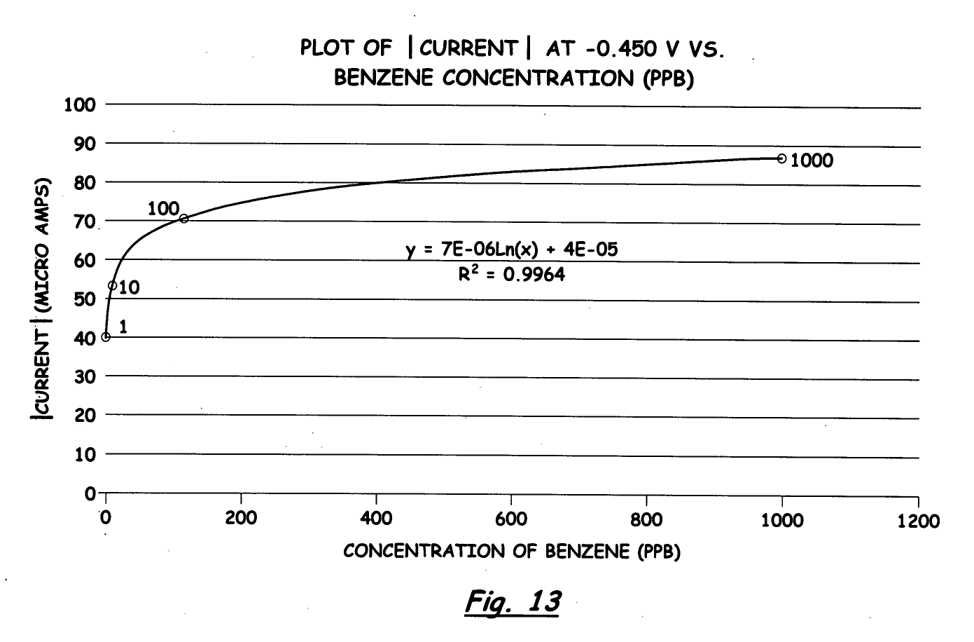

FIG. 13 shows the current plotted at −0.45 volts vs. benzene concentration in ppb, which may be described as y=7E-06Ln(x)+4E-05, with R²=0.9964.

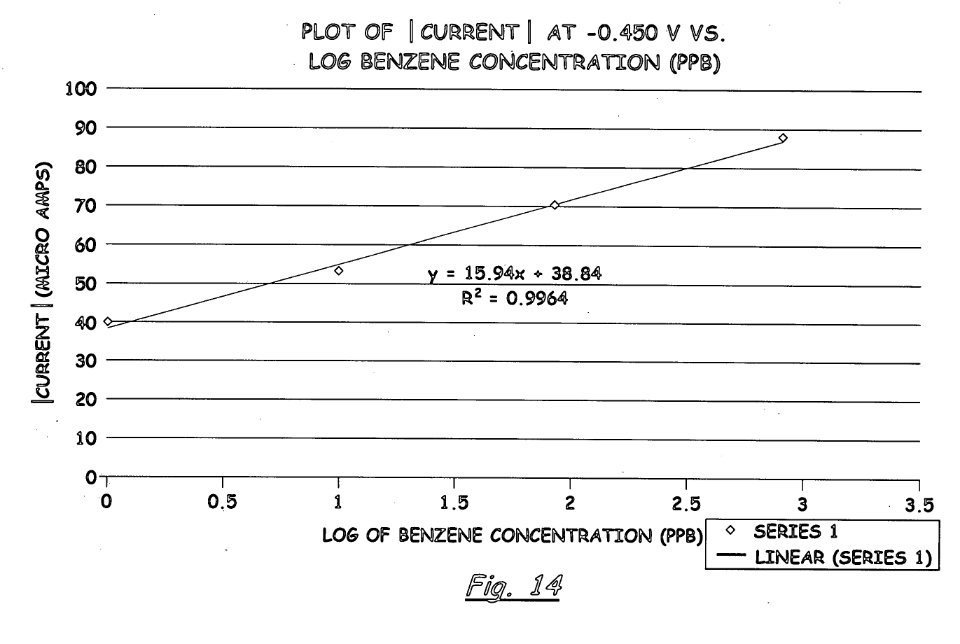

FIG. 14 shows this data plotted as current vs. log of benzene concentration, which may be described as y=15.94 x=38.84, with R²=0.9964.

Toluene:

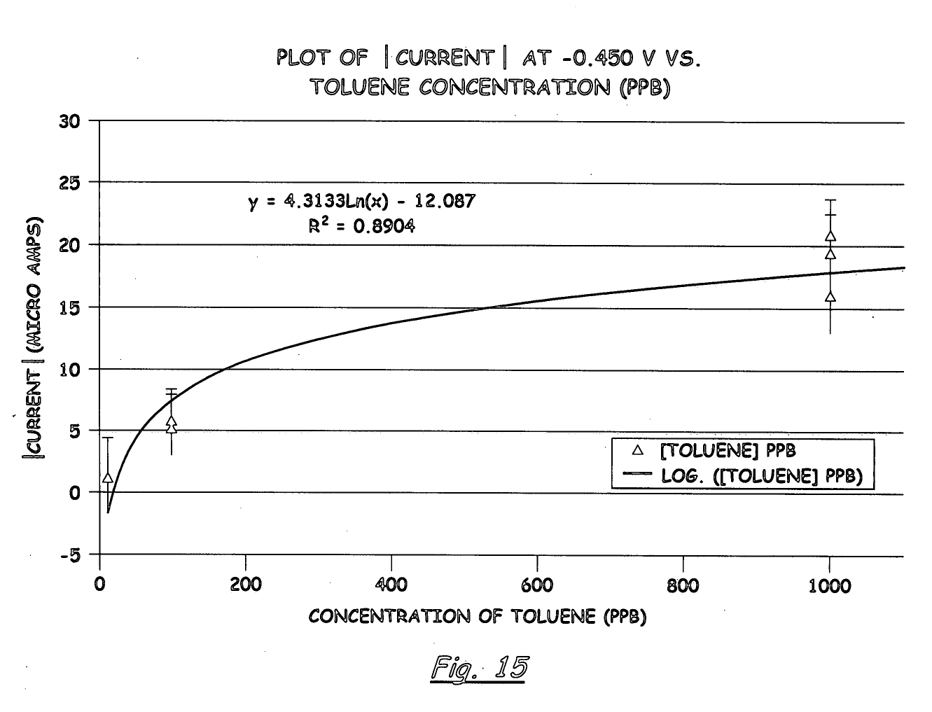

FIG. 15 shows testing of the same electrode in aqueous solutions containing various concentrations of toluene. The relationship of current to ppb toluene at −0.450 volts shown from this data may be described as y=4.3133Ln(x)−12.087, R²=0.8904.

Note that, while an adsorption isotherm based on concentration is expected to fit the data, simple linear equations have been found to also fit the data fairly well (FIGS. 13-15).

Catechol:

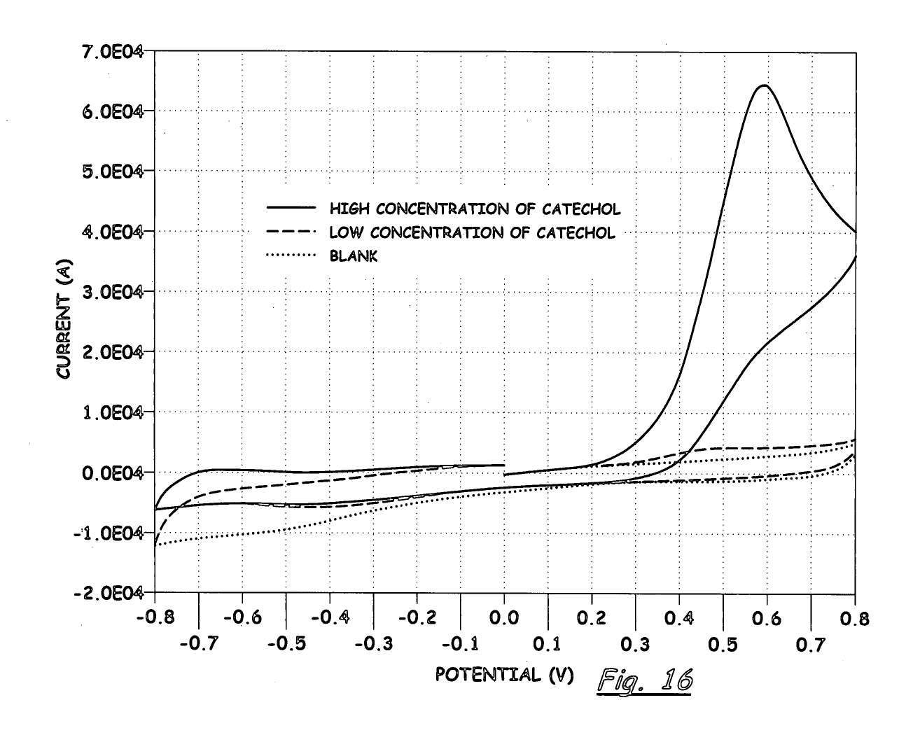

FIG. 16 shows testing of an electrode, manufactured by the same steps, in three aqueous solutions, one containing a high concentration of catechol, one containing a low concentration of catechol, and one being a blank solution without catechol. This data shows significant responses of the electrode to the catechol, compared to the blank, especially in the 0.3-0.8 volt potential range.

Embodiments of the invented apparatus and methods may be effective for detecting the presence, and measuring the amount present, of various benzenoid compounds in aqueous solutions, and even in vivo. The invented apparatus and methods are shown to be effective for benzene, toluene, and catechol, and are expected to be effective other benzenoid compounds, including those with fused benzene rings and polynuclear aromatics. Selectively of the apparatus and methods may be optimum for benzenoid compounds that have a benzene ring substantially exposed, that is, with only up to one side blocked by another ring(s) or other molecules. With the benzene more exposed, it is more available to fit selectively into the “hole” of the active binding site left for it by removal of the templating molecule.

Although this invention has been described above with reference to particular means, materials and embodiments, it is to be understood that the invention is not limited to these disclosed particulars, but extends instead to all equivalents within the scope of the following claims.

What we claim is:

1. An electrochemical sensor for benzene and benzenoid compounds, comprising:
a selective binding agent created by binding a template molecule comprising a benzene or benzenoid molecule with a monomer or dimer to arrive at a templated complex, polymerizing said templated complex, and removing the template molecule from the resulting polymer;
said selective binding agent being incorporated with an electrode that is adapted to detect changes in the conductivity of the binding agent in the presence of analyte benzene and benzenoid compounds.

2. The sensor of claim 1 wherein the binding agent is coated onto the electrode.

3. The sensor of claim 1 wherein the template molecule comprises benzene derivatized with active groups.

4. The sensor of claim 3 wherein said template molecule comprises benzene derivatized with active groups selected from the group consisting of: carboxylic acid groups, acid chloride groups, and active groups in meta relationship on carbons of the benzene.

5. The sensor of claim 4 wherein said template molecule is isophalaloyl dichloride comprising two Cl—C═O groups on benzene carbons in meta relationship.

6. The sensor of claim 5 wherein said monomer or dimer comprises 3-Thiophenemethanol.

7. The sensor of claim 6 wherein said polymerizing of the templated complex is done in the presence of thiophene dimer.

8. The sensor of claim 5 wherein said monomer or dimer comprises cyclopentane dithioiphene (CPDT).

9. The sensor of claim 8 wherein said polymerizing of the templated complex is done in the presence of excess CPDT.

10. The sensor of claim 1 wherein said binding a template molecule comprising a benzene or benzenoid molecule with a monomer or dimer comprises esterification and said removing the template molecule is done by reversing said esterification.

11. The sensor of claim 1 wherein said template molecule is benzene derivatized with two hydroxyl groups and said monomer or dimer comprises boron-derivatized thiophene.

12. The sensor of claim 1 wherein said template molecule is di-amine-derivatized benzene and said monomer or dimer comprises chloride-derivatized thiophene.

13. A method of sensing benzene or benzenoid compounds in a fluid, said method comprising:
forming a selective binding agent by steps comprising binding a template molecule comprising a benzene or benzenoid molecule with a monomer or dimer to arrive at a templated complex, polymerizing said templated complex, and removing the template molecule from the resulting polymer;
incorporating said selective binding agent with an electrode to form a sensor;
exposing said sensor to a fluid comprising benzene or benzenoid compounds and detecting changes in the conductivity of the binding agent in the presence of said benzene or benzenoid compounds in the fluid.

14. A method as in claim 13 wherein incorporating said selective binding agent with an electrode comprises deposition of said resulting polymer on said electrode during said polymerization.

15. The method of claim 13 wherein the template molecule comprises benzene derivatized with active groups selected from the group consisting of: carboxylic acid groups, acid chloride groups, and active groups in meta relationship on carbons of the benzene.

16. The method of claim 15 wherein said template molecule is isophalaloyl dichloride comprising two Cl—C═O groups on benzene carbons in meta relationship.

17. The method of claim 16 wherein said monomer or dimer comprises 3-Thiophenemethanol.

18. The method of claim 17 wherein said polymerizing of the templated complex is done in the presence of thiophene dimer.

19. The method of claim 16 wherein said monomer or dimer comprises cyclopentane dithioiphene (CPDT).

20. The method of claim 19 wherein said polymerizing of the templated complex is done in the presence of excess CPDT.

21. The method of claim 13 wherein said binding a template molecule comprising a benzene or benzenoid molecule with a monomer or dimer comprises esterification and said removing the template molecule is done by reversing said esterification.

22. The method of claim 13 wherein said removing of the template molecule is done by a mild acid wash.

23. The method of claim 13 wherein said removing of the template molecule is done by a mild base wash.

24. The method of claim 13 wherein said template molecule is benzene derivatized with two hydroxyl groups and said monomer or dimer comprises boron-derivatized thiophene.

25. The method of claim 13 wherein said template molecule is di-amine-derivatized benzene and said monomer or dimer comprises chloride-derivatized thiophene.

26. The method of claim 13 wherein said exposing said sensor to a fluid comprising benzene or benzenoid compounds comprises exposing said sensor to a fluid containing catechol.

27. The method of claim 13 wherein said exposing said sensor to a fluid comprising benzene or benzenoid compounds comprises exposing said sensor to a fluid containing catechol and ascorbic acid.

28. A method of detecting a non-electroreactive compound is a fluid with an electrochemical sensor, said method comprising:
providing a selective binding agent created by binding a template molecule comprising benzene with a monomer or dimer to arrive at a templated complex, polymerizing said templated complex, and removing the template molecule from the resulting polymer;
said selective binding agent being incorporated with an electrode that is adapted to detect changes in the conductivity of the binding agent in the presence of a non-electroreactive molecule; and
wherein said non-electroreactive molecule is benzene.

29. The method of claim 28 wherein said conductivity of the binding agent increases in the presence of the non-electroreactive molecule benzene.

30. The method of claim 28 wherein said template molecule comprises benzene derivatized with active groups.
